(19) United States
(12) Patent Application Publication
(10) Pub. No.: US 2009/0318403 A1
(43) Pub. Date: Dec. 24, 2009

(54) ANTIBIOTIC COMPOSITION

(75) Inventors: Peter Christian De Visser, Leiderdorp (NL); Gerard Johannes Platenburg, Voorschoten (NL); Jaap Tamino Van Dissel, Oegstgeest (NL)

Correspondence Address: MORGAN LEWIS & BOCKIUS LLP, 1111 PENNSYLVANIA AVENUE NW, WASHINGTON, DC, 20004, US

(73) Assignee: Prosensa Technologies B.V., Leiden (NL)

(21) Appl. No.: 12/376,221

(22) PCT Filed: Aug. 2, 2007
(86) PCT No.: PCT/NL2007/050387

§ 371 Date: May 19, 2009

(30) Foreign Application Priority Data
Aug. 3, 2006 (EP) 06118402.4

(51) Int. Cl. A61K 31/43; A61K 31/5377; A61K 31/4365; A61P 31/00; A61K 31/133; A61P 31/04; A61K 31/131; A01N 43/76; A01N 43/90

(52) U.S. Cl. 514/198; 514/236.8; 514/300; 514/663; 514/669

(57) ABSTRACT

The present invention provides antimicrobial compositions comprising an antibiotic and a sensitizer that enhances the effectiveness or activity of the antibiotic, wherein the sensitizer is preferably a primary amine containing a long alkyl chain. Such compositions are particularly useful for the treatment of infections by drug resistant bacteria.

### FIELD OF THE INVENTION

The present invention is in the field of antimicrobial compositions, in particular in the field of antimicrobial compositions comprising an antibiotic and a compound that acts as an enhancer of the antimicrobial effect of the antibiotic.

### BACKGROUND OF THE INVENTION

During the last decades a dramatic increase in bacterial strains that are resistant to one or more antibiotics has been reported. This increase has led to the occurrence of bacterial infections that can hardly or not at all be treated with the existing spectrum of antibiotics, which is a particularly serious problem in connection with e.g. hospital-acquired infections and has led to an increase in bacterial infections with a fatal outcome. The emergence of antibiotic resistance appears to be a result of the incorrect use of antibiotics in human and veterinary medicine combined with efficient bacterial mutation machinery.

The cell wall is a bacterial feature where particular antibiotic resistance originates, as it prevents many antibiotics from reaching their targets inside the cell, and may contain antibiotic efflux pump systems. Furthermore, bacteria may produce antibiotic hydrolyzing proteins (e.g. β-lactamases) that inactivate antibiotics. It is generally believed that if the bacterial membrane could only be rendered more permeable, the effect of antibiotics would be enhanced. Many attempts have been made to find effective ways of permeabilizing the bacterial outer membrane. Several polycations have been shown to permeabilize the outer membrane of Gram-negative bacteria, presumably by binding to the negatively charged lipopolysaccharide (LPS). Among the polycation permeabilizers are polymyxin B and its derivatives (see for example U.S. Pat. No. 4,510,132). Other polycationic permeabilizers include bactericidal/permeability-increasing protein, prolamine, and various polycationic and/or amphiphilic peptides including lysine oligomers, defensins, cecropins, magainins, and mellitin. Negatively charged chelators, such as ethylenediaminetetraacetate (EDTA), nitrilotriacetate, and sodium hexametaphosphate have also proved to be effective outer membrane permeabilizers, presumably by removing calcium and magnesium ions that cluster LPS units together, resulting in membrane destabilization. U.S. Pat. No. 6,165,997 discloses negatively charged phospholipids enhancing the activity of antimicrobials, but also having antimicrobial activity themselves. These compounds appear to act, at least partially, as the cation chelators described above.

JP 57155954 describes the enhancement of the activity of a basic peptide antibiotic substance in feed through the presence of a lipoamine consisting of 4 or more carbon atoms. The basic peptide antibiotic is based on amines, e.g. colistin A or B or polymyxin A, B, D or M. Disclosed lipoamines are monoamines, in particular butylamine, pentylamine, hexylamine, heptylamine, octylamine, nonylamine, decylamine, laurylamine and stearylamine. The focus is on suppressing the decrease of antibacterial activity caused by phospholipids, unsaturated fatty acids and calcium and magnesium present in livestock by addition of these amines to animal feed containing the antibacterial polymyxins. JP 57155954 shows that large amounts of lipoamines are required to restore or even improve antibacterial activity in feed, especially with respect to the amounts of antibacterial peptides present in the feed. Effects are only observed at molar ratios of stearylamine to colistin of at least 13:1. These relatively high concentrations of lipoamines would make them unattractive for use outside the field of animal feed.

Moreover, the lipoamines in JP 57155954 are carefully selected to match look-a-like polymyxins, resembling the hydrophobic tail which is essential for antibacterial activity itself. It is this structural resemblance that makes the skilled person expect some kind of synergistic action. Hence, at most the skilled person will regard the effect of butylamine up to stearylamine linked to those specific antibiotic peptides only.

WO-A-00/74654 discloses an administration form containing an acid-labile active compound in a matrix made of a mixture comprising triglyceride and solid paraffin. The mixture may contain further suitable excipients, such as polymers, sterols and basic compounds, among which stearylamine. Although WO-A-00/74654 suggests to combine the acid-labile active compound with antibiotics, there is no disclosure of such an administration form further containing antibiotics.

Similarly, U.S. Pat. No. 6,479,540 also teaches the use of stearylamine as a carrier. In the present case, the compositions contains tocol-soluble therapeutics, including antibiotics, as the active ingredients. Stearylamine is one of the many candidates to form an ion pair with the active ingredient, in order to increase its tocol solubility. No actual combination of an antibiotic and stearylamine is reported.

WO-A-00/30611 relates to compositions for treating protozoa, especially causative agents of malaria. It describes lipid vesicles which contain stearylamine, surrounding penicillin or tetracycline. Although no recipe is given, it suggests the use of large amounts of stearylamine, to form lipid vesicles. No hint on antibiotic resistance is given.

WO-A-04/00360 addresses the problem of developing resistance to antibiotics in treating dermatoses, and teaches the use of topical therapy. Stearylamine and dodecylamine are among the many possible basic compounds capable of producing a pH of 8.0 or greater in the topical formulation, thus acting as a skin permeation enhancer. Relatively large amounts are necessary for this purpose.

DE 10245506 describes formulations in which an active agent, such as an antibiotic, is embedded in a matrix of phospholipids and palmitoyl-D-glucuronide. The formulations are administered parenterally or by inhalation. The examples show that the amount of phospholipids largely exceeds that of the active ingredient.

### SUMMARY OF THE INVENTION

It is an objective of the present invention to provide antimicrobial compositions. It is further an objective to provide antimicrobial compositions that are active towards bacteria that are resistant to one or more antibiotics, in particular it is an objective to provide antimicrobial compositions that are active towards bacteria that are resistant to one or more antibiotics comprising the one or more antibiotics to which the bacteria are resistant. It is further an objective to provide antimicrobial compositions that have an enhanced activity of one or more antibiotics towards bacteria as compared to the antibiotic(s) alone.

It was found that certain long-chain alkylamines in combination with an antibiotic were capable of rendering bacteria susceptible to the antibiotic, whereas the antibiotic without the certain long-chain alkylamine was much less or not at all active against the bacteria.

Furthermore, it was found that long-chain alkylamines already promote antibiotic activity at concentrations significantly lower than those reported in the prior art.

Thus the invention concerns antimicrobial compositions comprising a long-chain amine of formula I or a physiologically acceptable salt thereof

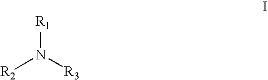

wherein
R₁ represents a linear or branched alkyl group comprising at least 7 atoms in a straight chain, said alkyl group may comprise double or triple bonds and may contain one or more substitutions, one or more cycloalkyl and/or aryl rings and may comprise one or more O, N and/or S atoms, and one of R₂ and R₃ is as is defined for R₁ and may be the same as R₁, or R₂ and R₃ are different from R₁, and R₂ and R₃ may be the same or different and represent a hydrogen or a lower alkyl group, said lower alkyl group may comprise double or triple bonds and may contain one or more substitutions or cycloalkyl and/or aryl rings and may comprise one or more O, N and/or S atoms, and the compositions further contain at least one antibiotic.

### DETAILED DESCRIPTION OF THE INVENTION

The present compositions comprise an antibiotic and a compound of formula I as defined above, wherein said compound of formula I acts as a sensitizer, meaning that it renders microorganisms, in particular bacteria, susceptible to the action of the antibiotic or that it renders said microorganisms susceptible to the action of the antibiotic at a lower concentration or dosage of the antibiotic. In particular it is found that bacteria that are resistant to certain types of antibiotics, or at least not affected by antibiotics at acceptable dosages, now effectively could be stopped proliferating or in fact killed at acceptable concentrations of the antibiotic when this antibiotic is administered in the presence of a sensitizer according to formula I.

Although it is immediately clear from the remainder of the text and the term “sensitizer”, it is emphasized that the compound according to formula I is an active ingredient in the composition. Hence, it is completely different from those situations where it serves as a carrier material, generally defined not to interact with other components of the composition. However, in the present case, the compound according to formula I is precisely included to enhance the activity of the antibiotic. This is reflected by the preferred (relative) amounts of the sensitizer.

R₁ in formula I represents a linear or branched alkyl group comprising at least 7 atoms in a straight chain. In this context alkyl group means that it is a group containing carbon atoms. For instance, for the lipoamines butylamine, pentylamine and hexylamine as disclosed in JP 57155954 no effect was found in our studies. It is understood that if not specified otherwise, C, O, N and S atoms in the alkyl group further comprise hydrogen atoms to properly satisfy the valency of the respective atom. At least 7 atoms in a straight chain is with respect to the longest group of atoms, not including hydrogen, directly connected to one another by covalent bonds starting from the nitrogen (N) in formula I. Such a group of at least 7 atoms in a straight chain thus can be part of cyclic elements, including aromatic rings, in R₁, or in other words, one or more (saturated and/or unsaturated) rings can form (part of) the alkyl group comprising at least 7 atoms in a straight chain. The alkyl group may comprise one or more O, N and/or S atoms, which means that the chain of carbon atoms may be interrupted by one or more O, N and/or S atoms. It is preferred the alkyl group comprises an ω-CH₃ group, or in other words starting from the nitrogen (N) in formula I the longest alkyl group preferably ends with a CH₃.

The R₁ alkyl group, in particular the at least 7 atoms containing straight chain, may comprise one or more double bonds or one or more triple bonds or combinations of double and triple bonds and/or cycloalkyl and/or aryl rings. In one embodiment the at least 7 atoms containing straight chain comprises one double bond.

Also the at least 7 atoms containing straight chain may be interrupted by one or more O, N and/or S atoms, yielding e.g. alkoxyalkyl, alkylthioalkyl, hydroxyalkyl, thioalkyl, aminoalkyl moieties and the like.

Also the at least 7 atoms containing straight chain may be substituted, for example by one or more halogen atoms and/or groups via one or more O, N and/or S atoms, e.g. hydroxyl, amine and/or thiol groups or O-, N- and/or S-lower (carboxy)alkyl or doubly bound O, N(—H or -alkyl) and/or S. Lower alkyl preferably means a group comprising 1-6 carbon atoms.

In one embodiment the at least 7 atoms containing straight chain alkyl group only contains carbon atoms. In one embodiment the straight chain alkyl group contains 7-30 carbon atoms, preferably 10-24 carbon atoms, more preferably 12-22 carbon atoms, most preferably at least 13 carbon atoms. In one embodiment the R₁ alkyl group only contains carbon atoms.

In one embodiment only one relatively large alkyl group, i.e. an at least 7 atoms containing straight chain alkyl group, is present in the sensitizer of formula I. In one embodiment R₂ and R₃ are different from R₁. It is thus preferred that R₂ and R₃ are selected from the group consisting of hydrogen and lower alkyl, wherein lower alkyl preferably is a group containing 1-6 carbon atoms.

The lower alkyl group may comprise one or more double bonds or one or more triple bonds or combinations of double and triple bonds and/or cycloalkyl and/or aryl rings.

Also the lower alkyl group may be interrupted by one or more or end with O, N and/or S atoms, e.g. alkoxyalkyl, alkylthioalkyl, hydroxyalkyl, thioalkyl, aminoalkyl and the like.

Also the lower alkyl group may be substituted, for example by one or more halogen atoms and/or groups via one or more O, N and/or S atoms, e.g. hydroxyl, amine and/or thiol groups or O-, N- and/or S-lower (carboxy)alkyl or doubly bound O, N(—H or -alkyl) and/or S. In one embodiment lower alkyl group means C1-C6 alkyl.

In one embodiment R₂ and R₃ are different and at least one of R₂ and R₃ is hydrogen. In another embodiment R₂ and R₃ are the same and preferably are selected from the group consisting of C1-C4 alkyl, preferably methyl, ethyl, propyl, isopropyl and butyl. In another embodiment R₂ and R₃ are the same and both are hydrogen.

In one embodiment the sensitizer of formula I is present in the form of a salt, in particular an acid addition salt, e.g. its HCl, HBr, HF, H₃PO₄, H₂SO₄, citric acid, acetic acid, trifluoroacetic acid, lactic acid, isethionic acid, methanesulfonic acid or ethylenediamine tetraacetic acid addition salt.

In the present compositions, preferably the sensitizer of formula I is present in an amount that is sufficient to enhance the effectiveness of the antibiotic.

By an effectiveness of an antibiotic is understood that the addition of the antibiotic to a culture medium inhibits growth of the inoculum such that the number of colony forming units (CFU) with the antibiotic is less than 30%, such as 20%, 15%, 10%, or 5% of the CFU without addition of the antibiotic. Preferably the addition of the antibiotic kills the inoculum such that the CFU is less than 70%, such as 60%, 50%, 40%, 30%, 20%, 10%, 5%, 2%, 1%, 0.1%, or 0.01% of the inoculum.

By an enhanced effect of an antibiotic is understood that the minimum inhibitory concentration (MIC) of the antibiotic without the sensitizer of formula I according to the present invention is decreased by at least 2-fold by the addition of said sensitizer. Preferably, the decrease is at least 4-fold, such as 8-fold, 10-fold or even more such as 20-fold, 50-fold or even 100-fold.

A person skilled in the art is able, based on routine experimentation, to determine what a suitable concentration of the sensitizer is, taking into account the particular sensitizer, the particular antibiotic and the extent to which enhanced effectiveness of the antibiotic is attained.

As reported above, it is part of the invention that the amounts of sensitizer need not be high to enhance the effectiveness of the antibiotic(s). It is preferred that the molar ratio of sensitizer(s) of formula I to antibiotic(s) is attractively lower than 5:1, more preferably lower than 2:1, most preferably lower than 1.5:1, particularly at most 1:1. More preferably, the molar ratio is at least 1×10⁻⁵:1, more preferably at least 5×10⁻⁵:1, most preferably at least 1×10⁻⁴:1.

Thus also the present compositions preferably comprise an antimicrobially, or antibiotically, effective amount of an antibiotic. From the above it follows that a skilled person is able, based on routine experimentation, to determine what a suitable concentration of the antibiotic is, taking into account the particular sensitizer, the particular antibiotic and the extent to which enhanced effectiveness of the antibiotic is attained.

In one embodiment the present composition comprises an antibiotic that is effective against Gram-negative bacteria. Gram-positive and Gram-negative bacteria are differentiated by the Gram stain. A Gram-positive species retains the primary stain (crystal violet) when treated with a decolourising agent (alcohol or acetone) whereas a Gram-negative bacterium loses the primary stain. The staining difference reflects the structural differences in the cell walls of Gram-negative and Gram-positive bacteria. The Gram-positive cell wall consists of a relatively thick peptidoglycan layer and teichoic acids whereas the Gram-negative cell wall consists of a relatively thin peptidoglycan layer, and an outer membrane consisting of a lipid bilayer containing phospholipids, lipopolysaccharide, lipoproteins and proteins.

In yet another embodiment the present composition comprises an antibiotic that is effective against Gram-positive bacteria.

In one embodiment the present composition comprises an antibiotic which is selected from the group consisting of β-lactams, (e.g. ampicillin, ceftazidime, meropenem), quinolones (e.g. norfloxacin, ciprofloxacin), glycopeptides (e.g. vancomycin), macrolides (e.g. erythromycin), oxazolidinones (e.g. linezolid), peptide antibiotics (e.g. magainin II), lipopeptides (e.g. polymyxins, bacitracin), nitroimidazoles (e.g. metronidazole), ansamycins (e.g. rifampin), azoles (e.g. fluconazole), D-cycloserine, lincosamides (e.g. clindamycin), mupirocin, streptogramins (e.g. dalfopristin, quinupristin), fosfomycin, aminoglycosides (e.g. gentamicin), sulfonamides (e.g. sulfomethoxazole), trimethoprim, tetracyclines (e.g. tigilcycline), novobiocin, chloramphenicol, monobactams and synthetic derivatives of these antibiotics.

More in particular, a (combination of) suitable antibiotic(s) to be used in combination with the sensitizer according to formula I is selected from the group consisting of glycopeptides (preferably vancomycin or teicoplanin), β-lactams, preferably penicillins, such as amdinocillin, ampicillin, amoxicillin, azlocillin, bacampicillin, benzathine penicillin G, carbenicillin, cloxacillin, cyclacillin, dicloxacillin, methicillin, mezlocillin, nafcillin, oxacillin, penicillin G, penicillin V, piperacillin, and ticarcillin; cephalosporins, such as the first generation drugs cefadroxil, cefazolin, cephalexin, cephalothin, cephapirin, and cephradine, the second generation drugs cefaclor, cefamandole, cefonicid, ceforanide, cefoxitin, and cefuroxime, or the third generation cephalosprins cefoperazone, cefotaxime, cefotetan, ceftazidime, ceftizoxime, ceftriaxone, and moxalactam; carbapenems such as imipenem; or monobactams such as aztreonam; further tetracyclines such as demeclocycline, tigilcycline, doxycycline, methacycline, minocycline, and oxytetracycline; aminoglycosides such as amikacin, gentamicin, kanamycin, neomycin, netilmicin, paromomycin, spectinomycin, streptomycin, and tobramycin; polymyxins such as colistin, colistimathate, and polymyxin B, and erythromycins and lincomycins and also sulfonamides such as sulfacytine, sulfadiazine, sulfisoxazole, sulfamethoxazole, sulfamethizole, and sulfapyridine; trimethoprim, quinolones, novobiocin, pyrimethamine, and rifampin are also expected to have enhanced activity in the presence of the sensitizer of formula I in a composition according to the present invention.

It is stressed that the sensitizer of the invention is not selected for its structural similarities with the antibiotic. It is found that the compound according to formula I also enhances the antimicrobial effect of the aforementioned antibiotics other than peptide antibiotics and lipopeptides, although having little in common structurally.

The invention also concerns a composition of a sensitizer of formula I as defined above and an antibiotic together with a pharmaceutical acceptable carrier. Such a pharmaceutical composition may be in solid, semi-solid, liquid etc. form, which are for internal or external application such as a tablet, capsule, liquor, vapour, ointment, paste, spray etc. Formulation into a suitable form is well known to a person skilled in the art, see e.g., “Remington's Pharmaceutical Sciences” and “Encyclopedia of Pharmaceutical Technology”.

For optimal interaction with the antibiotic, it is preferred that the sensitizer of formula I is not contained in a protective or embedding layer. The sensitizer of formula I and the antibiotic are preferably present in the same matrix.

Particularly preferred embodiments of the present invention are those wherein the sensitizer of formula I is selected from the group consisting of 3-lauryloxypropylamine, laurylamine, myristylamine, palmitylamine, stearylamine, eicosamine, oleylamine, linoleylamine, linolenylamine, sphingosine. In another preferred embodiments the compositions according to the present invention comprise the HCl salt of 3-lauryloxypropylamine, laurylamine, myristylamine, palmitylamine, stearylamine, eicosamine, oleylamine, linoleylamine, linolenylamine, sphingosine, dehydroabietylamine, or the citrate salt of tamoxifen. More particularly, the present invention comprises 3-lauryloxypropylamine, myristylamine, palmitylamine, eicosamine, oleylamine, linoleylamine, linolenylamine, sphingosine, dehydroabietylamine, tamoxifen, or the HCl salt thereof, or the citrate salt of tamoxifen.

In general the present compositions can be used to eradicate Gram negative and/or Gram positive bacteria from places where they are not desired. Thus the present invention also concerns the use of the present antimicrobial compositions for cleaning or sterilising of objects and areas. Preferably for this purpose the sensitizers of formula I as defined above and (an) antibiotic(s) and optionally further cleaning and/or sterilising agents are combined with a suitable carrier or diluent, for example such as water and/or (an) alcohol.

Animal feed formulations are not a preferred embodiment of the invention. It is more preferred that the composition of the invention is a pharmaceutical composition.

It is not part of the invention to provide novel administration forms for acid-labile active compounds. The composition of the invention is preferably free from such acid-labile active compounds, such as acid-labile proton pump inhibitors (H+/K+ ATPase inhibitors), in particular substituted pyridine-2-yl-methylsulfinyl-1H-benzimidazoles or prazoles. Alternatively or additionally, it is preferred not to use solid paraffin, such as paraffinum solidum (paraffin wax) or ozocerite in the composition.

The present invention also concerns the use of sensitizers of formula I as defined above and (an) antibiotic(s) for the preparation of a medicament for the treatment of a bacterial infection, preferably in human beings, particularly to enhance the antimicrobial activity of the antibiotic(s). Alternatively, the present invention also provides a method for treating bacterial infections in a patient in need thereof, preferably a human being, the method comprising administering the composition of the invention to the patient.

In one embodiment the medicament is used for the treatment of infections by bacteria that are resistant or multi-resistant to certain specific antibiotics. In one embodiment the medicament is used for the treatment of infection by Gram-negative bacteria. In one embodiment the medicament is used for the treatment of infections by Escherichia spp, in particular E. coli, Haemophilus spp, in particular H. influenzae, Pseudomonas spp, in particular P. aeruginosa, Klebsiella, in particular K. pneumoniae , Enterobacter spp., Helicobacter spp, in particular Helicobacter pylori, Shigella spp, Salmonella spp, Yersinia spp, Campylobacter spp, Neisseria spp, Bordetella spp, Aeromonas spp, Burkholderia spp, Serratia spp, Vibrio spp, Proteus mirabilis, and Acinetobacter spp, in particular A. baumannii.

In one embodiment the medicament is used for the treatment of infection by Gram-positive bacteria. In one embodiment the medicament is used for the treatment of infections by Staphylococcus spp, in particular methicillin-resistant Staphylococcus aureus, Streptococcus spp, Enterococcus spp, Listeria spp, Staphylococcus spp, Streptococcus spp, Clostridium spp, Bacillus spp, Enterococcus spp, Corynebacterium spp, Legionella spp, Mycobacterium spp,

Additionally, the invention also pertains to a kit of parts comprising:
a) at least one sensitizer represented by formula I; and
b) at least one antibiotic,
   intended for the treatment of a bacterial infection, particularly to enhance the antimicrobial effect of the antibiotic.

The sensitizer and the antibiotic may comprise one or more additional features as defined above. The kit of parts is intended for sequential or simultaneous administration, wherein the administration routes for the sensitizer and the antibiotic may be the same or different. Therein, it is preferred to adapt the amount of sensitizer administered to enhance the effectiveness of the antibiotic. Means for achieving this are described above.

### EXAMPLES

### Experimental Procedure for Assaying Sensitizers of Formula I

Solution

An 8 mM stock solution of long-chain alkylamine of formula I in ethanol was prepared for serial 2-fold dilution with ethanol. One equivalent of hydrochloric acid (from 1 M aqueous stock) was added to the first solution before dilution, in case the amine was not in the HCl-form. Tamoxifen citrate was dissolved in ethanol to provide the first 8 mM stock solution.

Bacteria

The concentration of an overnight culture (16 h, 37° C.) of Pseudomonas aeruginosa PA01 in 100% LB was determined by comparison with a calibration curve and diluted to 1×10⁵ CFU/mL with 100% LB. Likewise, cultures of the following clinical isolates (obtained at the Leiden University Medical Center) were prepared: multidrug-resistant Acinetobacter baumannii LUH5771, extended β-lactamase (ESBL) producing Klebsiella pneumoniae LUH5344 and Pseudomonae aeruginosa PA7243, PA7247, PA7249, PA7252, PA7253 and PA 24-7-3 (ceftazidime-resistant).

Test

Using 96-well plates, concentrations of sensitizer (1 μL of every ethanolic solution) were added to the wells containing 20 μL P. aeruginosa (final concentration 1×10⁴ CFU/mL, OD₅₅₀=0.1) and ampicillin or linezolid (in cases of PA 24-7-3 and ESBL K. pneumoniae LUH5344, ceftazidime was used). For 1×10⁴ CFU/mL A. baumannii LUH5771, the effect of sensitizers on growth was investigated in presence of gentamicin. The volume in the wells was adjusted to 200 μL with 20% LB. As controls, 20% LB, bacteria+20% LB, bacteria+20% LB+antibiotic were included. In some cases the medium was changed to 100% LB (vide infra) After addition of sensitizer at t=0, the 96-well plate was covered (not airtight) and incubated at 37° C. while shaking for 20 h in a BioTek plate reader; OD₅₅₀ was determined at least every 10 min for PA01 studies or once after 20 h for clinical isolates. A MIC value for a specific compound was determined as the lowest concentration at which, after 20 h incubation, the OD₅₅₀ value was comparable to that of the blank used.

Results

TABLE 1
Effect of sensitzers on the growth of P. aeruginosa
PA01 in 20% LB in presence of ampicillin or linezolid.

| Compounds | Effect |
|---|---|
| ampicillin | MIC >200 μg/mL |
| linezolid | MIC >200 ug/mL |
| oleylamine | MIC at ≧20 μM |
| 50 μg/mL ampicillin + oleylamine | MIC at ≧0.31 μM oleylamine |
| 200 μg/mL ampicillin + oleylamine | MIC at ≧0.08 μM oleylamine |
| 100 ug/mL linezolid + oleylamine | MIC at ≧5 μM oleylamine |
| stearylamine | MIC at ≧20 μM |
| 200 μg/mL ampicillin + stearylamine | MIC at ≧0.62 μM stearylamine |
| 100 ug/mL linezolid + stearylamine | MIC at ≧10 μM stearylamine |
| dehydroabietylamine | MIC at ≧20 μM |
| 200 μg/mL ampicillin + | MIC at ≧10 μM |
| dehydroabietylamine | dehydroabietylamine |
| tamoxifen | MIC at ≧20 μM |
| 200 μg/mL ampicillin + tamoxifen | MIC at ≧2.5 μM tamoxifen |

Representative examples of the sensitizers of formula I are oleylamine and stearylamine. As is shown in Table 1, these compounds render P. aeruginosa PA01 vulnerable to ampicillin. PA01 was killed by 200 μg/mL ampicillin in the presence of 0.62 μM stearylamine or 0.08 μM oleylamine. The growth of PA01 was also remarkably inhibited by linezolid in presence of sensitizer; linezolid is an antibiotic that is indicated only for treatment of Gram-positive species. PA01 was not only inhibited by ampicillin in combination with fatty amines, but also by combining ampicillin with the tricyclic dehydroabietylamine or anti-cancer agent tamoxifen.

Similarly, the effects of oleylamine as sensitizer were determined against clinical isolates of P. aeruginosa (Table 2). These isolates can be eradicated by the combination of ampicillin+oleylamine while being unaffected to high concentrations of both.

TABLE 2
Effect of oleylamine on the growth of clinical P. aeruginosa
isolates in presence of ampicillin.

| | | | Growth inhibiting (MIC) |
|---|---|---|---|
| | MIC | | compositions of ampicillin at |
| Isolate | ampicillin | oleylamine | concentrations oleylamine |
| PA7243 | >200 μg/mL | MIC at >40 μM | 200 μg/mL at ≧5 μM |
| | | | 50 μg/mL at ≧7.5 μM |
| PA7247 | >200 μg/mL | MIC at ≧40 μM | 200 μg/mL at ≧7.5 μM |
| | | | 50 μg/mL at ≧10 μM |
| PA7249 | >200 μg/mL | MIC at >40 μM | 200 μg/mL at ≧2.5 μM |
| | | | 50 μg/mL at ≧7.5 μM |
| PA7252 | >200 μg/mL | MIC at ≧40 μM | 200 μg/mL at ≧1.25 μM |
| | | | 50 μg/mL at ≧5 μM |
| PA7253 | >200 μg/mL | MIC at >40 μM | 200 μg/mL at ≧5 μM |
| | | | 50 μg/mL at ≧10 μM |

Inoculum 10⁴ bacteria in 20% LB, 20 h incubation.

Table 3 displays the effects of low concentrations of the sensitizers oleylamine and stearylamine on the MIC values of ampicillin to which PA01 normally is resistant; furthermore, Table 3 shows that the MIC value of the quinolone antibiotic nalidixic acid against PA01 is reduced upon addition of low concentrations of sensitizer. It should be noted that the MIC value of 25 μg/mL of an antibiotic is classified as clinically resistant.

TABLE 3
Effect of sensitzers on the MIC value of antibiotics
against P. aeruginosa PA01 (after 20 h).

| | MIC (μg/mL) |
|---|---|
| ampicillin | >200 |
| ampicillin + oleylamine 5 μM | 3.125 |
| ampicillin + stearylamine 3 μM | 6.25 |
| nalidixic acid | 25 |
| nalidixic acid + oleylamine 5 μM | 12.5 |
| nalidixic acid + oleylamine 20 μM | 6.3 |

Inoculum 10⁴ bacteria in 20% LB.

Furthermore, a multi-resistant Acinetobacter baumannii clinical isolate was found to be vulnerable to the action of gentamicin in the presence of oleylamine, whereas the species itself was resistant to treatment with gentamicin, ampicillin, the frequently used combination of gentamicin/ampicillin, or oleylamine alone (see Table 4).

TABLE 4
Effect of sensitizers on susceptibility of a multi-drug
resistant A. baumannii towards gentamicin.

| | MIC | | MIC of gentamicin at |
|---|---|---|---|
| | gentamicin | oleylamine | concentration oleylamine |
| AC | >200 μg · mL | bactericidal | 100 μg/mL at ≧0.62 μM |
| LUH5771 | | at ≧5 μM | |

Finally, use of oleylamine as sensitizer could lower the MIC value of ceftazidime significantly in cases of an extended β-lactamase producing K. pneumoniae and P. aeruginosa clinically isolated strains.

TABLE 5
Effect of compositions on extended-β-lactamase producing
K. pneumoniae clinical isolate LUH5344 and ceftazidime-resistant
P. aeruginosa clinical isolate PA 24-7-3.

| | MIC | | MIC ceftazidime + |
|---|---|---|---|
| | ceftazidime | oleylamine | oleylamine |
| ESBL | 6.25 μg/mL | MIC ≧40 μM | MIC <0.098 μg/mL |
| LUH5344 | | | at 20 μM oleylamine |
| PA 24-7-3 | 6.25 μg/mL | MIC ≧20 μM | MIC 1.56 μg/mL at |
| | | | 10 μM oleylamine |

(100% LB (LUH5344) or 20% LB (PA 24-7-3))

Similar results as depicted in the Tables above could be obtained using sensitizers with shorter R₁ groups (as depicted in formula I), such as laurylamine.

In the same manner as described above also methicillin-resistant Staphylococcus aureus (MRSA) is tested and is found to be sensitive towards methicillin when this is administered together with the long-chain alkylamines described above such as oleylamine and stearylamine. Likewise, vancomycin-resistant Enterococcus faecalis (VRE) can be killed with vancomycin and other glycopeptides in presence of sensitizers of formula I. Also, lower antibiotic dosages are found to be necessary for inhibiting growth of Staphylococcus spp, Streptococcus spp, Clostridium spp, Bacillus spp, Enterococcus spp, Corynebacterium spp, Legionella spp, Mycobacterium spp, Listeria spp in presence of sensitizers of formula I.

1. An antimicrobial composition comprising
a) a long-chain alkylamine of formula I or a physiologically acceptable salt thereof 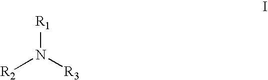
wherein
R₁ represents a linear or branched alkyl group comprising at least 7 atoms in a straight chain, said alkyl group may comprise double or triple bonds and may contain one or more substitutions, cycloalkyl or aryl rings, and may comprise one or more O, N and/or S atoms, and
R₂ and R₃ may be the same or different and represent a hydrogen or a lower alkyl group comprising 1-6 carbon atoms, said lower alkyl group may comprise double or triple bonds, cycloalkyl or aryl rings, and may contain one or more substitutions and may comprise one or more O, N and/or S atoms, and
b) at least one antibiotic,
wherein the molar ratio of a) to b) is lower than 5:1.

2. The antimicrobial composition according to claim 1, wherein said molar ratio of a) to b) is at most 1:1.

3. The antimicrobial composition according to any one of the preceding claims, wherein a) and b) are present in the same matrix.

4. The antimicrobial composition according to any one of the preceding claims, being a pharmaceutical composition.

5. The antimicrobial composition according to any one of the preceding claims, wherein the at least 7 atoms containing straight chain alkyl group only contains carbon atoms.

6. The antimicrobial composition according to any one of the preceding claims, wherein the straight chain alkyl group contains 12-22 carbon atoms.

7. The antimicrobial composition according to any one of the preceding claims, wherein the R₁ alkyl group only contains carbon atoms.

8. The antimicrobial composition according to any one of the preceding claims, wherein R₂ and R₃ represent hydrogen.

9. The antimicrobial composition according to any one of the preceding claims, wherein the long-chain alkylamine of formula I is in the form of its HCl salt.

10. The antimicrobial composition according to any one of the preceding claims, wherein the long chain amine is selected from the group consisting of laurylamine, myristylamine, palmitylamine, eicosamine, oleylamine, sphingosine, 3-lauryloxypropylamine, linoleylamine, linolenylamine, dehydroabietylamine, tamoxifen.

11. The antimicrobial composition according, to any one of the preceding claims, wherein the antibiotic is selected from β-lactams, quinolones, glycopeptides, macrolides, oxazolidinones, peptide antibiotics, lipopeptides, nitroimidazoles, ansamycins, azoles; D-cycloserine, -lincosamides, mupirocin, streptogramins, fosfomycin, aminoglycosides, sulfonamides, trimethoprim, tetracyclines, novobiocin, chloramphenicol, monobactams and synthetic derivatives thereof.

12. Use of a composition according to any one of the preceding claims for the preparation of a medicament for the treatment of a bacterial infection.

13. Use according to claim 12, to enhance the antimicrobial activity of the antibiotic(s)

14. Use according to claim 12 or 13 for the treatment of infection by Gram-negative bacteria.

15. Use according to claim 12-14 for the treatment of infections by Escherichia spp, Haemophilus spp, Pseudomonas spp, Klebsiella spp, Enterobacter spp., Helicobacter spp, Shigella spp, Salmonella spp, Yersinia spp, Campylobacter spp, Neisseria spp, Bordetella spp, Aeromonas spp, Burkholderia spp, Serratia spp, Proteus spp, Vibrio spp and Acinetobacter spp.

16. Use according to claim 12 for the treatment of infection by Gran-positive bacteria.

17. Use according to claim 12 or 16 for the treatment of infections by Staphylococcus spp. Streptococcus spp, Clostridium spp, Bacillus spp, Enterococcus spp, Corynebacterium spp, Legionella spp, Mycobacterium spp, and Listeria spp.

18. Use of the antimicrobial composition according to any one of claims 1-11, optionally comprising further cleaning and/or sterilising agents and optionally comprising a suitable carrier or diluent, for cleaning or sterilising of objects and areas.

19. Kit of parts comprising:
a) a long-chain alkylamine of formula I or a physiologically acceptable salt thereof 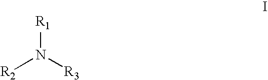
wherein
R₁ represents a linear or branched alkyl group comprising at least 7 atoms in a straight chain, said alkyl group may comprise double or triple bonds and may contain one or more substitutions, cycloalkyl or aryl rings, and may comprise one or more O, N and/or S atoms, and
R₂ and R₃ may be the same or different and represent a hydrogen or a lower alkyl group comprising 1-6 carbon atoms, said lower alkyl group may comprise double or triple bonds, cycloalkyl or aryl rings, and may contain one or more substitutions and may comprise one or more O, N and/or S atoms, and
b) at least one antibiotic,
wherein the molar ratio of a) to b) is lower than 5:1.

20. Use of the kit of parts according to claim 19 for the preparation of a medicament for the treatment of a bacterial infection.
